(19) United States
(12) Patent Application Publication
(10) Pub. No.: US 2014/0140634 A1
(43) Pub. Date: May 22, 2014

(54) RADIATION IMAGING APPARATUS, METHOD FOR CONTROLLING THE SAME, AND PROGRAM

(71) Applicant: Canon Kabushiki Kaisha, Tokyo (JP)

(72) Inventor: Shinsuke Hayashida, Yokohama-shi (JP)

(21) Appl. No.: 14/084,802

(22) Filed: Nov. 20, 2013

(30) Foreign Application Priority Data
Nov. 21, 2012 (JP) 2012-255426

(51) Int. Cl. H04N 5/30; G06T 5/00

(52) U.S. Cl. CPC H04N 5/30; G06T 5/002
USPC 382/264; 250/336.1

(57) ABSTRACT

A radiation imaging apparatus includes a radiation imaging unit configured to capture a radiation image of a subject, a wireless communication unit configured to perform wireless communication with an external apparatus, a wireless reception characteristics obtaining unit configured to obtain a wireless reception characteristic of the wireless communication unit, an estimation unit configured to estimate an amount of noise included in the radiation image on the basis of a characteristic value of the wireless reception characteristic, and a correction unit configured to correct the noise included in the radiation image on the basis of the amount of noise estimated by the estimation unit.

### BACKGROUND OF THE INVENTION

1. Field of the Invention

The present invention relates to a radiation imaging apparatus that captures a radiation image of a subject, a method for controlling the radiation imaging apparatus, and a program for causing a computer to execute the method. Although an example in which X-rays are used as radiation will be taken herein, the radiation to be used is not limited to the X-rays and, for example, electromagnetic waves, alpha rays, beta rays, gamma rays, or the like may be used, instead.

2. Description of the Related Art

In these years, digital X-ray imaging apparatuses that perform imaging using, for example, imaging units that use photoelectric conversion elements are gaining in popularity in X-ray imaging for medical diagnosis. Because the digital X-ray imaging apparatuses are capable of obtaining X-ray transmission images without developing films or the like, the digital X-ray imaging apparatuses achieve more instantaneity than when films or the like are used.

Current X-ray imaging apparatuses transmit and receive various pieces of signal information, that is, the current X-ray imaging apparatuses not only transmit image information obtained using imaging units to hospital information systems but also transmit and receive information regarding timing at which X-rays are generated to and from X-ray generation apparatuses and receive driving information from the hospital information systems.

In these years, between X-ray imaging apparatuses and X-ray generation apparatuses or hospital information systems, not only wired communication for which dedicated lines are used but also wireless communication in which X-ray images are synchronized and communication of imaging is performed is possible.

For example, in U.S. Pat. No. 7,856,085, an accumulation time switching unit that switches accumulation time between an X-ray generation apparatus in accordance with a wired/wireless communication method used in X-ray imaging and an X-ray imaging apparatus and a reduction process unit that reduces increased dark current noise caused by increased accumulation time are disclosed.

In U.S. Patent Application Publication No. 2006/0244627, a technique for limiting operability in a certain period of time using wireless reception characteristics is disclosed.

In an X-ray imaging apparatus capable of performing wireless communication, noise in an image might increase during the wireless communication. In particular, for example, when a wireless communication unit necessary for the wireless communication is disposed close to an imaging unit, small noise generated by the wireless communication unit might be undesirably superimposed upon an X-ray image that has not been subjected to amplification, thereby mixing the noise into the X-ray image.

### SUMMARY OF THE INVENTION

A radiation imaging apparatus in the present invention includes a radiation imaging unit configured to capture a radiation image of a subject, a wireless communication unit configured to perform wireless communication with an external apparatus, a wireless reception characteristics obtaining unit configured to obtain a wireless reception characteristic of the wireless communication unit, an estimation unit configured to estimate an amount of noise included in the radiation image on the basis of a characteristic value of the wireless reception characteristic, and a correction unit configured to correct the noise included in the radiation image on the basis of the amount of noise estimated by the estimation unit.

In addition, the present invention includes a method for controlling the radiation imaging apparatus and a program for causing a computer to execute the method.

Further features of the present invention will become apparent from the following description of exemplary embodiments with reference to the attached drawings.

### BRIEF DESCRIPTION OF THE DRAWINGS

FIG. 1 is a diagram illustrating an example of the schematic configuration of an X-ray imaging system (radiation imaging system) according to a first embodiment of the present invention.

FIG. 2 is a flowchart illustrating an example of a processing procedure of a control method used by the X-ray imaging system (radiation imaging system) according to the first embodiment of the present invention.

FIGS. 3A-1 to 3B are conceptual diagrams illustrating communication performed by an X-ray imaging apparatus (radiation imaging apparatus) according to embodiments of the present invention.

FIG. 4 is a diagram illustrating an example of a schematic configuration relating to calibration according to a second embodiment of the present invention.

FIG. 5 is a flowchart illustrating an example of a processing procedure of a control method used by an X-ray imaging system (radiation imaging system) according to the second embodiment of the present invention.

FIG. 6 is a diagram illustrating the calibration according to the second embodiment of the present invention.

FIGS. 7A to 7D are diagrams illustrating a threshold used in a line noise correction process according to the embodiments of the present invention.

FIGS. 8A to 8C are diagrams illustrating a filtering process relating to a noise correction process according to the embodiments of the present invention.

FIG. 9 is a flowchart illustrating an example of a processing procedure of a control method used by an X-ray imaging system (radiation imaging system) according to a third embodiment of the present invention.

### DESCRIPTION OF THE EMBODIMENTS

Modes for implementing the present invention (embodiments) will be described hereinafter with reference to the drawings.

### First Embodiment

First, a first embodiment of the present invention will be described.

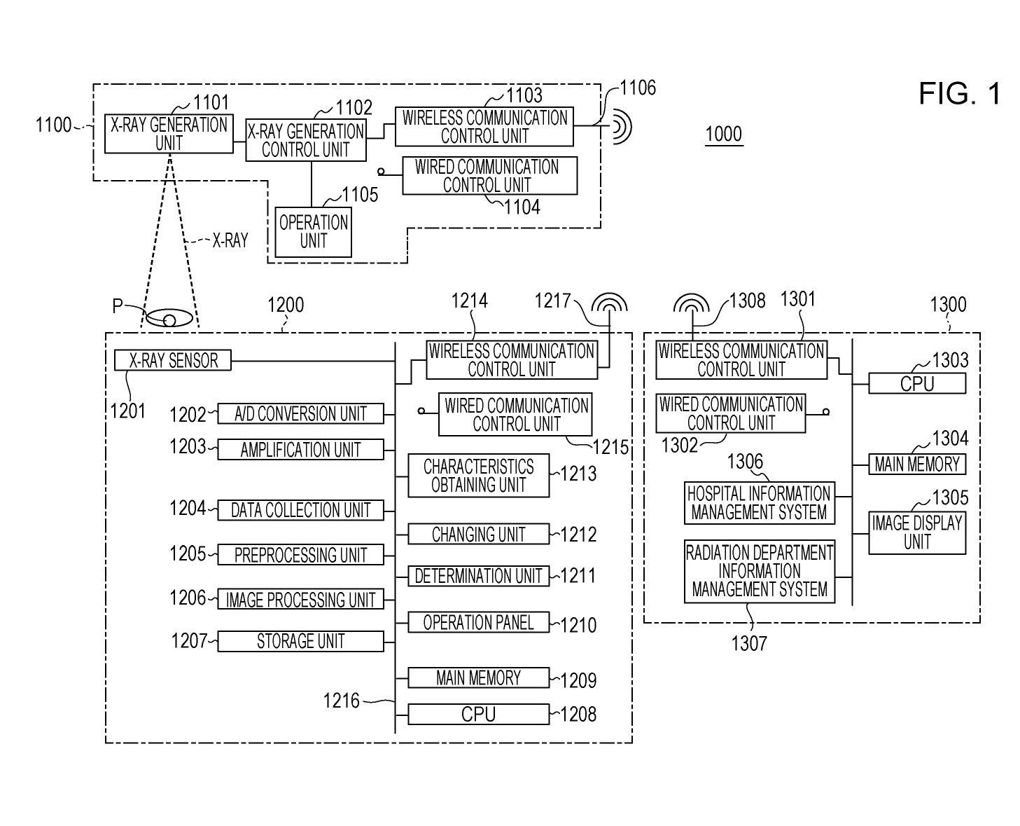

FIG. 1 is a diagram illustrating an example of the schematic configuration of an X-ray imaging system (radiation imaging system) according to the first embodiment of the present invention.

As illustrated in FIG. 1, an X-ray imaging system 1000 according to this embodiment includes an X-ray generation apparatus (radiation generation apparatus) 1100, an X-ray imaging apparatus (radiation imaging apparatus) 1200, and a hospital information system 1300. Here, an X-ray generation unit (radiation generation unit) 1101 included in the X-ray generation apparatus 1100 and an X-ray sensor (radiation sensor) 1201 included in the X-ray imaging apparatus 1200 are arranged in such a way as to face each other with a subject P disposed therebetween.

The X-ray generation apparatus 1100 is an apparatus that generates X-rays while, for example, communicating with an external apparatus or the like. The X-ray generation apparatus 1100 includes the X-ray generation unit 1101, an X-ray generation control unit (radiation generation control unit) 1102, a wireless communication control unit 1103, a wired communication control unit 1104, an operation unit 1105, and a wireless communication unit 1106.

The X-ray generation unit 1101 is connected to the X-ray generation control unit 1102, and radiates X-rays onto the subject P on the basis of control performed by the X-ray generation control unit 1102.

The X-ray generation control unit 1102 is connected to the X-ray generation unit 1101, the wireless communication control unit 1103, the wired communication control unit 1104, and the operation unit 1105. For example, the X-ray generation control unit 1102 controls the X-ray generation unit 1101 on the basis of information from the wireless communication control unit 1103, the wired communication control unit 1104, or the operation unit 1105.

The wireless communication control unit 1103 controls wireless communication between the X-ray generation apparatus 1100 and an external apparatus or the like. The wireless communication control unit 1103 is connected to the wireless communication unit (wireless antenna or the like) 1106 for performing the wireless communication between the X-ray generation apparatus 1100 and the external apparatus or the like. The wired communication control unit 1104 controls wired communication between the X-ray generation apparatus 1100 and an external apparatus or the like through a wired communication unit.

The operation unit 1105 is operated by a user (operator) when, for example, the user issues an instruction or the like to the X-ray generation apparatus 1100.

The X-ray imaging apparatus 1200 captures an X-ray image based on X-rays that have passed through the subject P. The X-ray imaging apparatus 1200 includes the X-ray sensor 1201, an analog-to-digital (A/D) conversion unit 1202, an amplification unit 1203, a data collection unit 1204, a preprocessing unit 1205, an image processing unit 1206, a storage unit 1207, a central processing unit (CPU) 1208, a main memory 1209, an operation panel 1210, a determination unit 1211, a changing unit 1212, a characteristics obtaining unit 1213, a wireless communication control unit 1214, a wired communication control unit 1215, a CPU bus 1216, and a wireless communication unit 1217.

In the X-ray sensor 1201, pixels each including a conversion element that converts X-rays that have passed through the subject P into an electrical signal (image signal) and a switching element for transferring the electrical signal, such as a thin-film transistor (TFT), are arranged in two dimensions. Here, for example, each conversion element includes a scintillator that converts X-rays into light and a photoelectric conversion element that converts the light obtained as a result of the conversion performed by the scintillator into an electrical signal (image signal), or each conversion element is formed by an element that directly converts X-rays into an electrical signal (image signal).

The X-ray sensor 1201 is connected, through the CPU bus 1216, to the A/D conversion unit 1202, the amplification unit 1203, the data collection unit 1204 that collects X-ray image data (radiation image data), and the preprocessing unit 1205 that preprocesses the X-ray image data obtained by the data collection unit 1204. Furthermore, the CPU bus 1216 is connected to the image processing unit 1206, the storage unit 1207 that stores X-ray image data, the CPU 1208, the main memory 1209, and the operation panel 1210. Furthermore, the CPU bus 1216 is connected to the determination unit 1211, the changing unit 1212, and the characteristics obtaining unit 1213. Furthermore, the CPU bus 1216 is connected to the wireless communication control unit 1214 that controls wireless communication with the X-ray generation apparatus 1100 and the hospital information system 1300 and the wired communication control unit 1215 that controls wired communication with the X-ray generation apparatus 1100 and the hospital information system 1300. In addition, the wireless communication control unit 1214 is connected to the wireless communication unit (wireless antenna or the like) 1217 for performing wireless communication between the X-ray imaging apparatus 1200 and an external apparatus or the like.

The main memory 1209 stores various pieces of data necessary for processing performed by the CPU 1208, and functions as a working memory for the CPU 1208. The CPU 1208 controls, for example, the operation of the entirety of the X-ray imaging apparatus 1200 in accordance with operations performed on the operation panel 1210 using the main memory 1209.

When the user (operator) has input an imaging instruction through the operation panel 1210, the CPU 1208 performs control for saving the content of the imaging instruction to the storage unit 1207 and control for displaying the content of the imaging instruction on the operation panel 1210.

Thereafter, when the user (operator) has issued an instruction to generate X-rays using the operation unit 1105 of the X-ray generation apparatus 1100, the X-ray generation apparatus 1100 controls the X-ray generation unit 1101 through the X-ray generation control unit 1102 in order to radiate X-rays onto the subject P and perform X-ray imaging.

In the X-ray imaging, the X-rays radiated from the X-ray generation unit 1101 pass through the subject P while being attenuated, and then reach the X-ray sensor 1201, which outputs an X-ray image signal.

The X-ray image signal output from the X-ray sensor 1201 is converted into a certain digital signal by the A/D conversion unit 1202 and fed (transmitted) to the preprocessing unit 1205 as X-ray image data. A main function of the preprocessing unit 1205 is to correct the characteristics of the X-ray imaging apparatus 1200. More specifically, for example, the preprocessing unit 1205 performs, for the X-ray image data, a gain correction process in which variations in the sensitivity of pixels of the X-ray sensor 1201 are corrected and a dark current correction process in which variations in the dark current of the pixels are corrected. When performing the correction processes, the preprocessing unit 1205 performs preprocessing by reading, as necessary, a gain correction image and a dark current correction image used for the correction that have been stored in the main memory 1209 before the user (operator) begins the X-ray imaging. Furthermore, the preprocessing unit 1205 performs, as necessary, a noise correction process (noise suppression process) on the X-ray image data as preprocessing. The X-ray image data subjected to the preprocessing performed by the preprocessing unit 1205 is transferred to the main memory 1209 and the image processing unit 1206 through the CPU bus 1216 as original image data by control performed by the CPU 1208.

Communication is performed by the wireless communication control unit 1214 or the wired communication control unit 1215, and when the wired communication control unit 1215 is performing wireless communication, components such as the characteristics obtaining unit 1213 is not used. On the other hand, when the wireless communication control unit 1214 is performing wireless communication, the characteristics obtaining unit 1213 performs a process for obtaining wireless reception characteristics.

In an X-ray imaging apparatus capable of performing wired and wireless communication, a wireless communication unit is invalidated while the wired communication is being performed, and therefore noise is not mixed into an X-ray image. On the other hand, while the wireless communication is being performed, the wireless communication unit is turned on and a wireless module undesirably receives wireless signals in close frequency bands. The amount of noise may be reduced to a certain extent by performing control such that the wireless communication is not performed while X-rays are being radiated onto a subject and an amplifier in an imaging unit is amplifying electrical signals based on the X-rays that have passed through the subject.

If activation time and deactivation time of the wireless communication unit are taken into consideration, however, it is not desirable in terms of the workflow of the X-ray imaging to stop the wireless communication performed by the wireless communication unit while an X-ray image is being obtained because it takes time to generate an image signal and a synchronization signal for synchronization with an X-ray generation apparatus. In addition, if the degree of noise correction remains constantly high, the linear structure of image signals is affected. Therefore, the degree of correction needs to be kept to minimum in accordance with the amount of noise.

The determination unit 1211 determines a method (correction method) used in the noise correction process, the degree of the noise correction process, and the like in accordance with the wireless reception characteristics obtained by the characteristics obtaining unit 1213.

For example, if it is determined that there are a large number of wireless signals in close frequency bands, an image in which the subject P is easy to recognize may be obtained by changing the content of the noise correction process. The changing unit 1212 performs a process for changing the noise correction process performed by the preprocessing unit 1205 and the image processing unit 1206. For example, the changing unit 1212 changes the method (correction method) used in the noise correction process, the degree of the noise correction process, and a threshold used in the noise correction process performed by the preprocessing unit 1205 and the image processing unit 1206 by changing external variables of the preprocessing unit 1205 and the image processing unit 1206.

After the preprocessing unit 1205 corrects the characteristics of the X-ray imaging apparatus 1200 and the like, the image processing unit 1206 performs, on the X-ray image data, which is the original image data, image processing such as a frequency process, the noise correction process (noise suppression process), and a tone process according to a display medium such as a monitor or a film. Thereafter, for example, the image processing unit 1206 transmits the X-ray image data subjected to the image processing to the hospital information system 1300 through the wireless communication unit 1217. The hospital information system 1300 displays an X-ray image based on the X-ray image data transmitted from the X-ray imaging apparatus 1200 on an image display unit 1305.

If the wireless communication control unit 1214 receives wireless signals while an image is being read, noise might be superimposed upon the image. In particular, if there are wireless signals in close frequency bands, the wireless communication control unit 1214 might receive the wireless signals. As a method for directly obtaining whether or not there is interference in wireless communication in the surroundings, for example, software for checking a frequency channel interference state in wireless communication may be used. For example, pieces of software called inSSIDer (registered trademark) and OmniPeek (registered trademark) may be used. As an indirect method, it is possible to indirectly obtain interference in wireless communication by checking the strength (speed and reception sensitivity) of the wireless communication.

Because the noise correction process in the image processing is not optimized to a situation where there are a large number of wireless signals in frequency bands close to those used in ordinary environments of wired and wireless communication, the content of the noise correction process may be appropriately changed if the effect of a surrounding wireless communication environment upon the image noise can be obtained by monitoring the surrounding wireless communication environment.

As a wireless communication method used by the wireless communication unit 1217, one of various known communication methods may be used, such as Bluetooth (registered trademark), a high-speed wireless access network type a (HiSWANa), a high performance radio local area network (HiperLAN), Wireless 1394, and a wireless local area network (LAN).

The hospital information system 1300 includes a wireless communication control unit 1301, a wired communication control unit 1302, a CPU 1303, a main memory 1304, the image display unit 1305, a hospital information management system 1306, a radiation department information management system 1307, and a wireless communication unit 1308. The wireless communication control unit 1301 is connected to the wireless communication unit (wireless antenna or the like) 1308 for performing wireless communication between the hospital information system 1300 and an external apparatus or the like.

Although a configuration example in which the X-ray generation apparatus 1100, the X-ray imaging apparatus 1200, and the hospital information system 1300 are capable of performing wired and wireless communication with one another is illustrated in FIG. 1, a configuration in which these apparatuses are capable of performing only wireless communication may be adopted in the present invention.

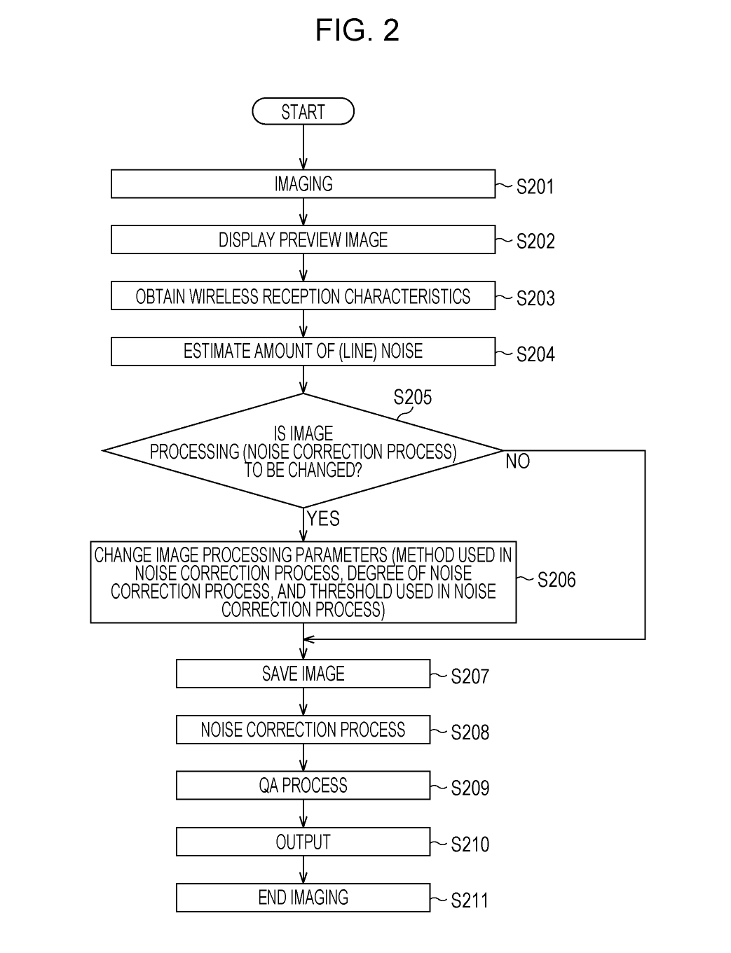

FIG. 2 is a flowchart illustrating an example of a processing procedure of a control method used by the X-ray imaging system (radiation imaging system) 1000 according to the first embodiment of the present invention. In FIG. 2, an example of a processing procedure of a control method used by the X-ray imaging apparatus 1200 is mainly illustrated.

First, in step S201, the X-ray imaging system 1000 performs X-ray imaging of the subject P. In the X-ray imaging, the X-ray generation unit 1101 of the X-ray generation apparatus 1100 radiates X-rays onto the subject P, and the X-ray sensor 1201 of the X-ray imaging apparatus 1200 converts the distribution of the X-rays that have passed through the subject P into an electrical signal (image signal) for each pixel. The X-ray imaging apparatus 1200 including the X-ray sensor 1201 is connected to the X-ray generation apparatus 1100 and the hospital information system 1300 through wired or wireless communication, and configured in such a way as to be able to transmit and receive timing signals and image signals (image data). X-ray image data obtained as a result of the X-ray imaging performed in step S201 is transmitted from the X-ray imaging apparatus 1200 to the hospital information system 1300. In step S201, imaging for obtaining a dark image in a state in which X-rays are not radiated is also performed in addition to the X-ray imaging, in order to correct variations in dark current and a residual image for each pixel.

Next, in step S202, the hospital information system 1300 displays an X-ray image based on the X-ray image data received from the X-ray imaging apparatus 1200 on the image display unit 1305 as a preview image. Here, the X-ray image obtained by the X-ray imaging in step S201 is displayed on the image display unit 1305 as a preview image in several seconds. The preview image is used for checking whether or not the X-ray imaging has been correctly performed, and is instantaneously displayed while performing almost no process for analyzing the X-ray image.

Next, in step S203, the characteristics obtaining unit 1213 of the X-ray imaging apparatus 1200 obtains information indicating the wireless reception characteristics of the wireless communication unit 1217. Here, a period from when the X-ray sensor 1201 generates the image signals to when the amplification unit 1203 performs an amplification process is a period in which noise caused by wireless communication can affect the X-ray image the most with noise. In the flowchart of FIG. 2, the process for obtaining the wireless reception characteristics (S203) is performed after the display of the preview image (S202), but the order may be reversed in the present invention. That is, the wireless reception characteristics may be obtained immediately after the X-ray image is obtained, which is before the display of the preview image.

Next, in step S204, for example, the determination unit 1211 (or the CPU 1208) of the X-ray imaging apparatus 1200 performs a process for estimating the amount of noise in the X-ray image obtained in step S201. Here, the wireless reception characteristics includes, for example, communication speed, channel interference, received signal strength indication (or received signal strength indicator; RSSI), and the sensitivity of the wireless module. If, for example, the characteristic value (signal strength value) of RSSI is larger than a certain value (−50 dBm or the like), it is determined that the sensitivity is higher than a threshold, and the noise correction process is not changed. If, for example, the characteristic value (signal strength value) of RSSI is smaller than the certain value, that is, if the sensitivity is lower than the threshold, however, the wireless communication characteristics are compared with values recorded before shipment in order to estimate the amount of noise included in the X-ray image. If the characteristic value of RSSI is equal to the certain value, the noise correction process may be but need not be changed.

The wireless reception sensitivity decreases, for example, for the following reasons in addition to wireless interference: a distance to a wireless transmission apparatus is large; the wireless transmission power of the wireless transmission apparatus and a wireless reception apparatus is low; the maximum value of wireless output level is restricted to a small value; and the remaining charge of a battery of each apparatus is low. Although a term “noise” is used here, estimation of the amount of line noise is also obviously included in the scope of the present invention because noise caused by an external factor is often line noise due to characteristics at a time when an image is read.

Next, in step S205, for example, the determination unit 1211 (or the changing unit 1212) of the X-ray imaging apparatus 1200 determines whether or not to change the noise correction process performed by the preprocessing unit 1205 and the image processing unit 1206 on the basis of the wireless reception characteristics obtained in step S203 (furthermore, the amount of noise estimated in step S204 on the basis of the wireless reception characteristics).

The preview image has already been displayed on the image display unit 1305 in step S202. Therefore, if it is determined that the noise is not to be reduced, that is, for example, if it is determined that the degree of the line noise correction process is not to be increased because the subject P has a linear structure, the noise correction process is not changed even if the wireless reception characteristics are lower than the threshold. In addition, because the preview image is generally a reduced image, step S205 may be performed again as necessary after an image used for diagnosis is output to the monitor or the film.

In this embodiment, the degree of the noise correction process and the threshold used in the noise correction process are changed in accordance with the wireless reception characteristics (furthermore, the amount of noise estimated on the basis of the wireless reception characteristics), and these changes are desirable because such changes are simple in terms of installed software. In terms of image quality, that is, more particularly, in terms of separation of subject signals from noise components, however, the method (correction method) used in the noise correction process can also be changed.

Here, a case in which the threshold used in the noise correction process is changed will be described.

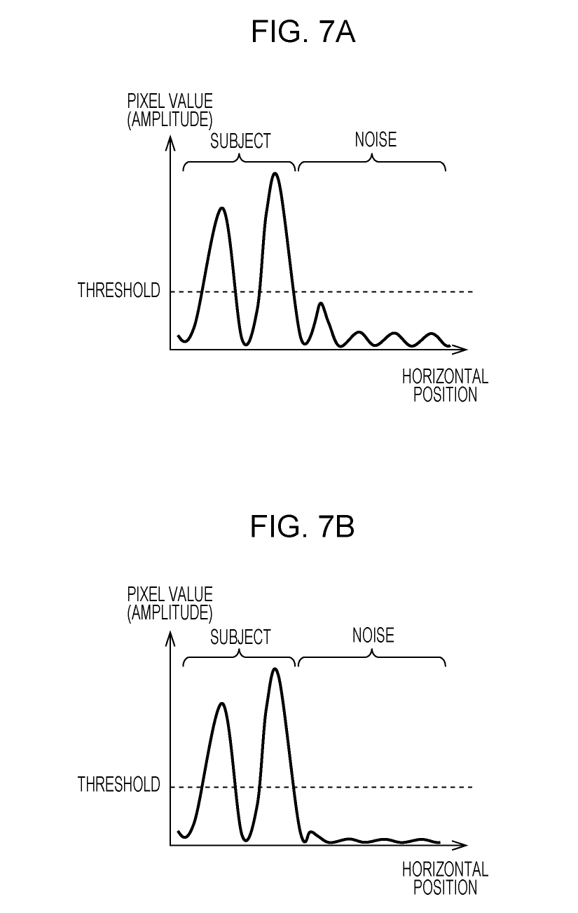

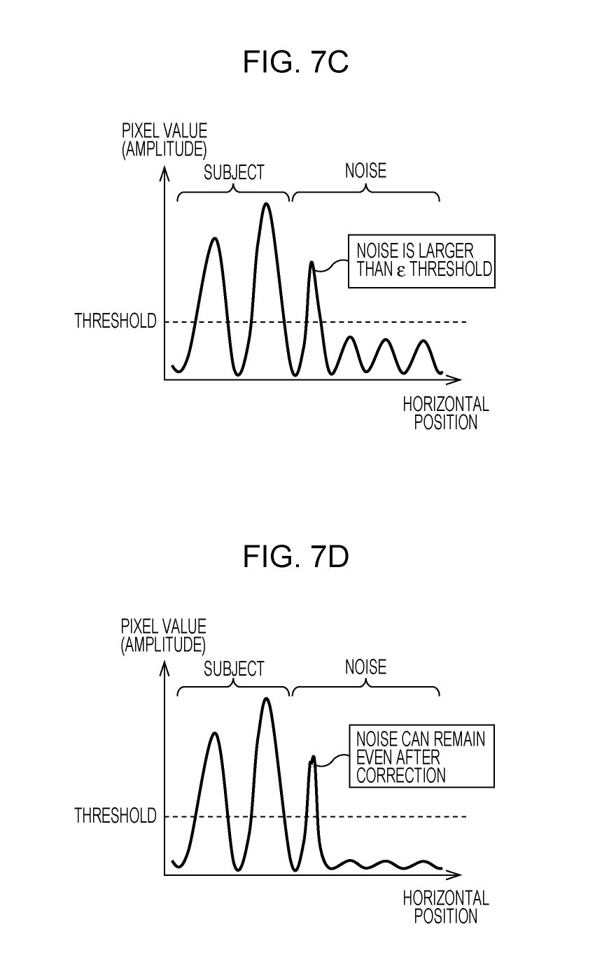

FIGS. 7A to 7D are diagrams illustrating the threshold used in the line noise correction process according to embodiments of the present invention. In the embodiments of the present invention, a method used in the line noise correction process or the like in which, among pixels arranged in a certain direction, pixels that exceed a certain threshold are removed from a line noise image may be used.

FIGS. 7A and 7B are diagrams illustrating image signals at a time when wired communication is being performed or wireless interference is not occurring. In FIGS. 7A and 7B, subject information and noise are appropriately separated from each other using a predetermined threshold, which makes it possible to reduce only noise components.

FIGS. 7C and 7D are diagrams illustrating image signals at a time when, for example, wireless interference is occurring. Noise is so large that a line noise component is as large as subject information. Since noise is larger than an ε threshold, the line noise component undesirably remains in a subject image as illustrated in FIG. 7D if an ε filtering correction process is performed using the same threshold as that used in the wired communication or the like illustrated in FIG. 7B. Therefore, in the present invention, by changing the degree of the noise correction process, that is, for example, by changing the threshold, a subject image in which the subject P is easy to recognize may be obtained while suppressing a possibility that the subject image is overcorrected. More specifically, in the example illustrated in FIG. 7D, for example, the degree of the noise correction process may be increased by increasing the threshold. In this case, for example, the degree of the noise correction process may be increased by increasing the threshold as the amount of noise increases.

Although a case in which the threshold used in the noise correction process is changed has been described as an example of a change to the noise correction process in this embodiment, the scope of the present invention is not limited to this. For example, as will be described later, a process for reducing noise components at certain frequencies in accordance with the wireless reception characteristics using a multiple frequency process or a filtering process such as one that uses a notch filter is also included in the scope of the present invention.

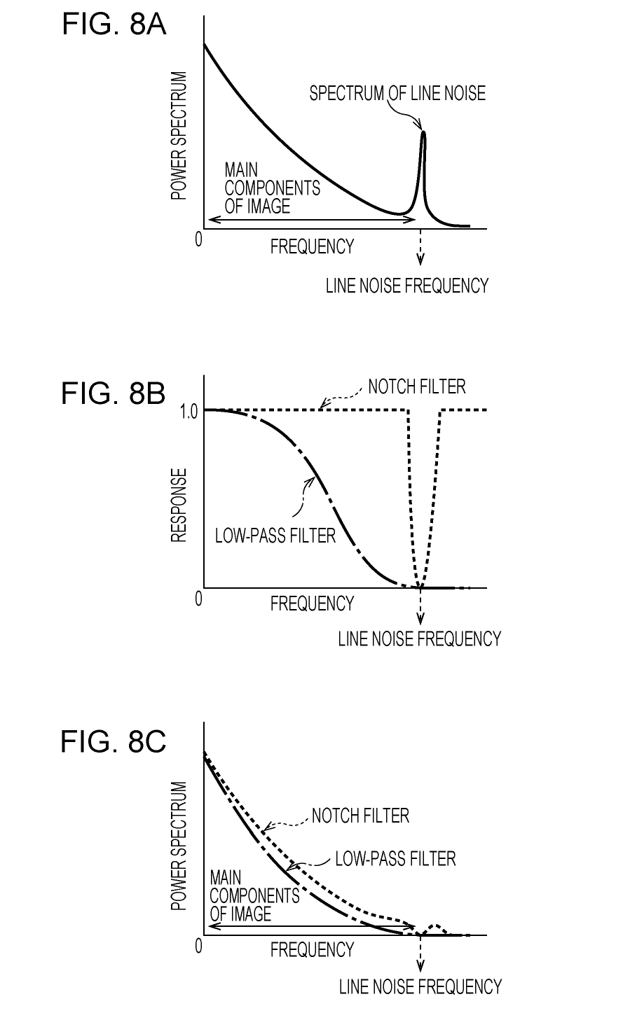

FIGS. 8A to 8C are diagrams illustrating a filtering process relating to the noise correction process according to the embodiments of the present invention.

In the case of noise having a certain period, a periodic process is more effective than the process illustrated in FIGS. 7A to 7D. FIG. 8A illustrates a power spectrum representing generated noise in an image. FIG. 8B illustrates filtering characteristics for reducing noise in a certain frequency band. FIG. 8C illustrates a power spectrum representing the amount of noise after the original image is subjected to the filtering process.

Noise in an image correlated with the wireless reception characteristics includes not only noise generated by electromagnetic waves of wireless signals but also electromagnetic noise generated when a wireless module that has received a wireless signal performs an operation different from a normal operation and noise caused by variations in power supply voltage and ground voltage. Therefore, since noise in an image is not necessarily periodic noise, the determination unit 1211 according to the embodiments of the present invention can obtain whether or not image noise is periodic, and change the (correction) method (the method used in the process illustrated in FIGS. 7A to 7D and the method used in the process illustrated in FIGS. 8A to 8C) used in the noise correction process.

If it is determined as a result of the determination made in step S205 that the noise correction process is to be changed, the procedure proceeds to step S206.

In step S206, the changing unit 1212 of the X-ray imaging apparatus 1200 performs a process for changing image processing parameter, that is, the method (correction method) used in the noise correction process, the degree of the noise correction process, and the threshold used in the noise correction process. Here, for example, the degree of the noise correction process is increased or the line noise correction process is performed at more frequencies as the amount of noise estimated in step S204 increases, in order to reduce noise. However, since diagnostic performance is affected if subject information is also reduced, only noise is reduced.

When the processing in step S206 has been completed or if it is determined in step S205 that the noise correction process is not to be changed, the procedure proceeds to step S207.

In step S207, for example, the CPU 1208 of the X-ray imaging apparatus 1200 performs a process for saving the X-ray image data that has not been subjected to the preprocessing performed by the preprocessing unit 1205 to the storage unit 1207. Since the amount of data of a still image is generally smaller than that of a moving image, not only the X-ray image data that has not been subjected to the preprocessing but also X-ray image data subjected to the preprocessing can obviously be saved. By saving the X-ray image data that has not been subjected to the preprocessing, it becomes possible to perform processing again even after an irreversible noise correction process such as the ε process is performed.

Next, in step S208, the preprocessing unit 1205 and the image processing unit 1206 of the X-ray imaging apparatus 1200 perform the noise correction process (noise suppression process) on the X-ray image data saved in step S207 on the basis of the current image processing parameters (the method (correction method) used in the noise correction process, the degree of the noise correction process, and the threshold used in the noise correction process). That is, here, the noise correction process is performed on the basis of the amount of noise estimated in step S204.

Next, in step S209, the preprocessing unit 1205 and the image processing unit 1206 of the X-ray imaging apparatus 1200 perform a quality assurance (QA) process on the X-ray image data subjected to the noise correction process in step S208. Since the noise and the like have been appropriately corrected in step S208, certain portions of the image are made more visible in step S209 by performing the tone process and the frequency process for the purpose of visual diagnosis.

Next, in step S210, the X-ray imaging apparatus 1200 performs a process for outputting the X-ray image data subjected to the QA process in step S209 to, for example, the hospital information system 1300. As a result, for example, the image display unit 1305 of the hospital information system 1300 displays an X-ray image based on the X-ray image data.

Thereafter, in step S211, the X-ray imaging apparatus 1200 performs a process for ending the X-ray imaging.

The processing procedure of the control method used by the X-ray imaging system 1000 illustrated in FIG. 2 thus ends.

Next, the wireless reception characteristics according to the embodiments of the present invention will be described. Not only a method for directly obtaining the wireless reception characteristics but also a method for indirectly obtaining the wireless reception characteristics is included in the scope of the present invention.

For example, interference at close frequencies, the sensitivity of the wireless module, and the like can be checked as the wireless reception characteristics on the basis of the channel interference, the communication speed, and the RSSI (received signal strength indication). In direct measurement of the wireless interference, the channel interference may be used, and in indirect measurement of the wireless interference, the communication speed and the RSSI may be used. The RSSI is an indicator for measuring the strength of a signal received by a wireless communication apparatus. The RSSI is mainly used for controlling the transmission range in wireless communication such as a wireless LAN or Bluetooth. This is because if the power of a wireless communication apparatus is too high, the transmission range covers unnecessary locations distant from the wireless communication apparatus, thereby undesirably consuming much power. As in the case of a mobile telephone, the operation panel 1210 displays a measured value for an antenna symbol (indicates the sensitivity level using the number of lines) indicating the reception sensitivity on the basis of the RSSI.

Regardless of whether an image is being obtained or calibration is being performed, the characteristics obtaining unit 1213 obtains the wireless reception characteristics such as a media access control (MAC) address, an extended service set identifier (ESSID; or a service set identifier (SSID)), a channel, the channel interference, the RSSI (received signal strength identification), a security method, the sensitivity of the wireless module, and the communication speed. The wireless reception characteristics include obtaining of the characteristic value of the set sensitivity (gain) of the wireless module. In general, it is difficult to adjust the power of a wireless module on a transmission side in accordance with an environment and conditions in wireless standards typified by Bluetooth and ZigBee (registered trademark). The wireless reception sensitivity of a wireless module on a reception side is usually set to a certain value on the basis of power consumption, but the set sensitivity (gain) of the wireless module may be often changed in accordance with a communication distance and output power. In many cases, the amplification function of such a wireless module is configured by capacitors or the like, and the sensitivity (gain) of the wireless module is changed by selecting the number of capacitors from the outside. Needless to say, direct measurement of the set sensitivity (gain) of a wireless module is included in the scope of the present invention.

Next, a relationship between frequencies in wireless communication and communication interference will be briefly described.

Wireless interference occurs, for example, in a frequency band at about 2.4 GHz among wireless frequency bands. This frequency band is often used in communication such as wireless LAN technologies and Bluetooth according to IEEE 802.11b and IEEE 802.11g. A 5 GHz band refers to a band at about 5 GHz among frequency bands according to a wireless LAN standard. This band is adopted in an IEEE 802.11a standard, which defines a high-speed wireless communication technology, and is rapidly put into practice in order to realize high-speed wireless communication.

In wireless LAN communication, the frequency band at about 2.4 GHz has been used. However, because the 2.4 GHz frequency band may be used by any device without permission insofar as power used is low, there is a problem in that communication speed might decrease if there is a microwave oven or a Bluetooth device nearby. On the other hand, the 5 GHz band does not pose such a problem.

The 5 GHz band is not often used by electric devices, but air traffic control radars, meteorological radars (e.g. Automated Meteorological Data Acquisition System (AMeDAS)), and the like use the 5 GHz band. Therefore, wireless LAN devices that use the 5 GHz band are obliged to incorporate a technology called “dynamic frequency control (DFC)” for automatically avoiding interference with such radars. However, it is difficult to stop other radars and wireless devices, and it might not be possible for the DFC to find a frequency at which there is no interference even if the DFC can find one of a plurality of wireless frequencies at which interference is the smallest. Wireless LANs that use the 5 GHz band had not been allowed to be used outdoors in Japan, but since January 2007, the wireless LANs have been allowed to be used outdoors at channels added as a result of revision of a ministerial ordinance.

In wireless communication, an identifier (ID) called an “ESSID” is often assigned for identification of a unit. In this case, a wireless LAN access point relays only communication with a terminal having the same ESSID. Therefore, even in an environment in which a plurality of wireless LAN access points exist at close positions, unnecessary communication such as crosstalk is avoided. In practice, however, when the wireless communication control unit 1214 has received a signal for checking the ESSID, small noise is superimposed upon an analog signal that has not been amplified, thereby mixing the noise into an image. That is, the wireless communication control unit 1214 might receive a signal transmitted in wireless communication in order to check an authentication ID such as the ESSID. The process performed by the characteristics obtaining unit 1213 basically assumes wireless interference, but the scope of the present invention is not limited to this. For example, obtaining only whether or not wireless communication or wired communication is being performed and, in the case of the wireless communication, performing control such that noise in an image is strongly corrected regardless of the wireless reception characteristics is also included in the scope of the present invention.

FIGS. 3A-1 to 3B are conceptual diagrams illustrating communication performed by the X-ray imaging apparatus 1200 according to the embodiments of the present invention. More particularly, the reason why noise increases in the X-ray imaging apparatus 1200 when there is interference in a wireless channel and when the RSSI (received signal strength indication) decreases will be described.

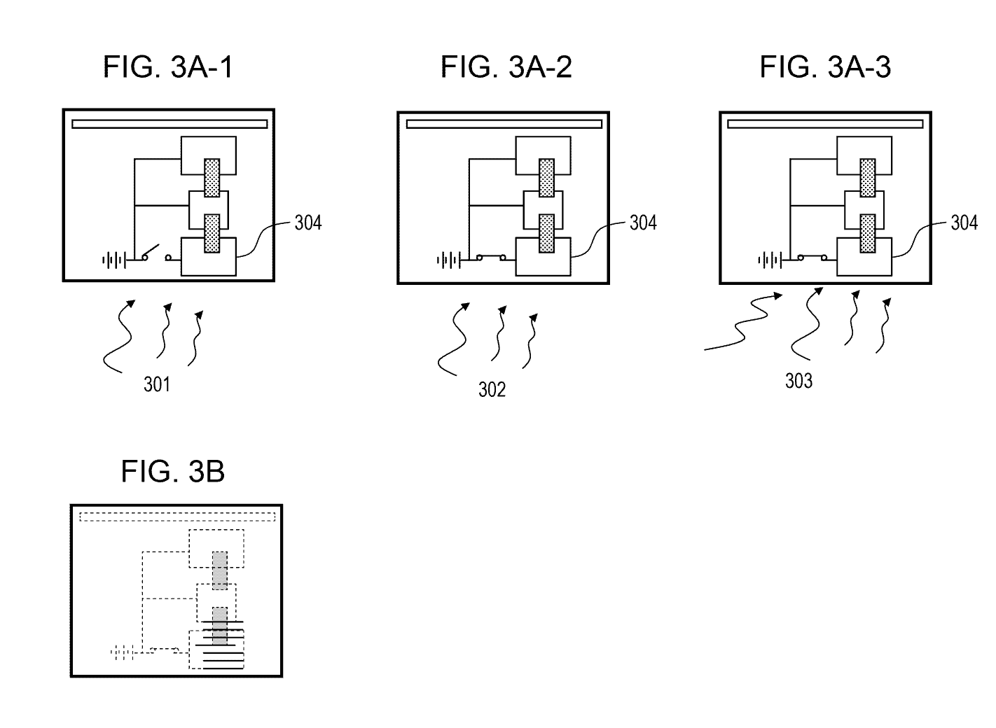

Thick frames illustrated in FIGS. 3A-1 to 3A-3 indicate a case of the X-ray imaging apparatus 1200, and a wireless module 304 including the wireless communication control unit 1214 and the wireless communication unit 1217 is provided inside the case. In the X-ray imaging apparatus 1200 illustrated in FIG. 3A-1, the wireless module 304 is not connected to a power supply and is performing wired communication. In the X-ray imaging apparatus 1200 illustrated in FIGS. 3A-2 and 3A-3, the wireless module 304 is connected to the power supply and is performing wireless communication.

In FIGS. 3A-1 to 3A-3, external wireless states 301 to 303, respectively, under these conditions are illustrated. Here, a difference between the external wireless state 302 illustrated in FIG. 3A-2 and the external wireless state 303 illustrated in FIG. 3A-3 is the degree of interference in external wireless communication.

In the external wireless state 302 illustrated in FIG. 3A-2, there are no wireless signals in frequency bands significantly close to the frequencies of wireless signals used by the wireless module 304 in the wireless communication. In this case, the wireless module 304 does not receive wireless signals in the external wireless state 302. Therefore, in the case illustrated in FIG. 3A-2, an external wireless state does not virtually exist.

On the other hand, in the external wireless state 303 illustrated in FIG. 3A-3, there is another wireless device that performs wireless communication using wireless signals (hereinafter referred to as “other wireless signals”) in frequency bands significantly close to the frequencies of the wireless signals used by the wireless module 304 in the wireless communication. In this case, the wireless module 304 determines whether or not to perform communication after receiving the other wireless signals once in order to determine whether or not to receive the other wireless signals using SSIDs or cryptographic keys. Therefore, small current flows into the wireless module 304 and, depending on the timing, electromagnetic noise generated by the wireless module 304 might affect the X-ray imaging apparatus 1200, thereby superimposing noise upon an image signal that is being subjected to a reading operation in this period of time before amplification. This is why noise increases in the X-ray imaging apparatus 1200 when there is interference in a wireless channel. When the RSSI (received signal strength indication) decreases, too, the amount of current increases so that the sensitivity of the wireless module 304 increases and wireless reception becomes possible. Therefore, electromagnetic noise increases due to current generated by the wireless module 304, thereby superimposing noise upon an image signal in the X-ray imaging apparatus 1200.

FIG. 3B illustrates a relationship between the structure inside the case of the X-ray imaging apparatus 1200 and an image. More specifically, FIG. 3B is a diagram illustrating strong line noise superimposed upon a region of the wireless module 304 at a time when the image is superimposed upon the structure inside the case of the X-ray imaging apparatus 1200.

It is possible that line noise increases in certain regions of the image when line noise is superimposed upon an image signal that is being subjected to the reading operation in the period of time described with reference to FIG. 3A-3 before the amplification. Therefore, in the embodiments of the present invention, the method used in the line noise correction is particularly changed.

As an example of the method used in the line noise correction, a threshold process unit may perform a threshold process on a line noise image in a certain direction (the same direction as the direction of the line noise). Because line noise has a strong correlation in a horizontal direction, the threshold process unit may adopt a method in which pixels are compared in the horizontal direction and subject pixels are removed from the line noise image.

In the embodiments of the present invention, for example, the degree of the line noise correction (the degree of suppression) and the threshold used in the line noise correction may be changed for each region of the image as necessary using the above-described technique in accordance with the wireless reception characteristics. At this time, in the embodiments of the present invention, the amount of noise is estimated in each region of the image.

### Second Embodiment

Next, a second embodiment of the present invention will be described.

The second embodiment is characterized in that the amount of noise is estimated more accurately by calibrating a wireless state before imaging.

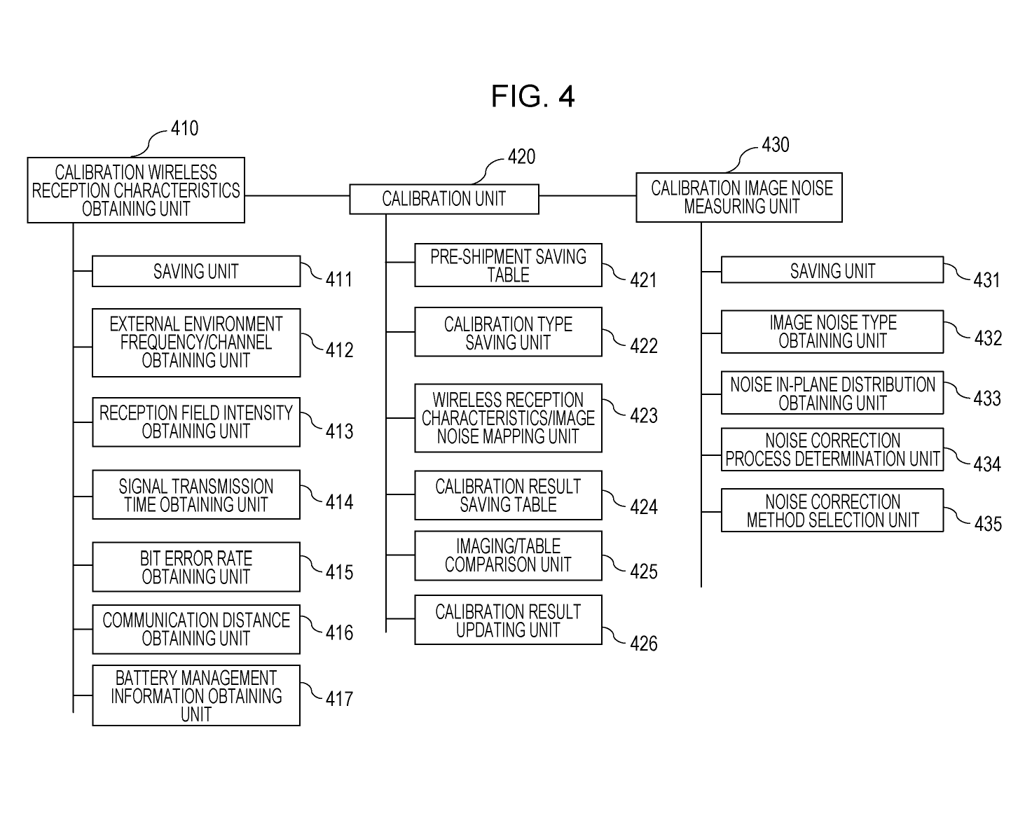

FIG. 4 is a diagram illustrating an example of a schematic configuration relating to the calibration according to the second embodiment of the present invention. The configuration illustrated in FIG. 4 is a configuration added to the inside of the X-ray imaging apparatus 1200 illustrated in FIG. 1.

In this embodiment, a table for calibrating a relationship between the wireless state and noise in an image are provided in advance, and the relationship between the wireless state and the noise in the image is obtained in an actual environment before the imaging and in an actual use environment during the calibration.

A calibration wireless reception characteristics obtaining unit 410 and a calibration image noise measuring unit 430 are linked to each other by a calibration unit 420.

The calibration wireless reception characteristics obtaining unit 410 includes a saving unit 411, an external environment frequency/channel obtaining unit 412, a reception field intensity obtaining unit 413, a signal transmission time obtaining unit 414, a bit error rate obtaining unit 415, a communication distance obtaining unit 416, and a battery management information obtaining unit 417.

The calibration unit 420 calibrates the relationship between the wireless reception characteristics and the noise (estimated amount of noise or the like) in the image. The calibration unit 420 includes a pre-shipment saving table 421, a calibration type saving unit 422, a wireless reception characteristics/image noise mapping unit 423, a calibration result saving table 424, an imaging/table comparison unit 425, and a calibration result updating unit 426.

The calibration image noise measuring unit 430 includes a saving unit 431, an image noise type obtaining unit 432, a noise in-plane distribution obtaining unit 433, a noise correction process determination unit 434, and a noise correction method selection unit 435.

The wireless reception characteristics that may be applied in this embodiment may be divided into two categories: wireless reception characteristics such as the RSSI and characteristics correlating with indirect wireless reception such as information regarding a distance between wireless communication units (wireless communication apparatuses) and the remaining charge of a battery. Because the two categories need not be strictly distinguished from each other for the calibration, applying both the categories is included in the scope of the present invention.

Here, in particular, calibration relating to the information regarding the distance between the wireless communication units (wireless communication apparatuses) and battery management information will be described.

First, the information regarding the distance between the wireless communication units (wireless communication apparatuses) will be described.

It is known that the wireless reception characteristics change in accordance with the physical distance between the wireless communication units (wireless communication apparatuses). Because the wireless reception characteristics can be not only a direct cause but also an indirect cause of generation of noise in an image, an error becomes large if the noise in the image is estimated or calculated using only the wireless reception characteristics. Therefore, it is highly necessary to obtain the distance between the wireless communication unit on the transmission side that performs wireless communication (wireless communication apparatus on the transmission side) and the wireless communication unit on the reception side (wireless communication apparatus on the reception side) during both the calibration and the X-ray imaging. Since the degree of deterioration in the wireless reception characteristics caused by the distance may be obtained, it is possible to accurately estimate the obtained wireless reception characteristics and the amount of noise in the image.

Next, the battery management information will be described.

In battery management, the wireless reception characteristics inevitably change. The battery management includes, for example, not only management of the remaining charge of a battery and battery voltage but also management of the power of a wireless signal transmission side (includes a wireless base station and a router). For example, a maximum value (maximum power) of wireless transmission power (transmitter power (current power)) is set and the power level of an associated client device (client power (dBm)) is restricted.

An apparatus that transmits and receives wireless signals, such as the X-ray imaging apparatus 1200, often includes a battery (power supply), and it is empirically possible that when the remaining charge of the battery or the battery voltage has fallen below a certain value, the wireless reception characteristics deteriorate. Furthermore, the wireless power of a transmission apparatus might not be constant, which affects the wireless reception characteristics. This is because the maximum value of the wireless transmission power of the transmission apparatus is set and the power level is restricted in order for transmission of unnecessarily strong wireless electromagnetic waves not to affect the operations of various electric devices and the health of humans. With respect to these pieces of information used in an environment in which a reception apparatus and a transmission apparatus are used in a hospital or the like, the wireless reception characteristics and the amount of noise may be accurately estimated by obtaining the battery information regarding the reception apparatus and the transmission apparatus and performing the calibration in substantially the same environment in advance.

Furthermore, by saving the battery management information obtained during the calibration and, before capturing an X-ray image, monitoring whether or not the wireless reception characteristics have changed, it is possible to obtain whether or not to update a result of the calibration. If the monitored wireless reception characteristics are significantly different from those obtained during the calibration, a calibration updating unit that updates the result of the calibration can be included.

Although the operations of the communication distance obtaining unit 416 that obtains the information regarding the distance between the wireless communication units and the battery management information obtaining unit 417 have been described in this embodiment, wireless reception characteristics obtaining units that may be applied in the present invention are not limited to these. For example, a unit that obtains the RSSI, the signal transmission time obtaining unit 414 that obtains signal transmission time in wireless communication, the reception field intensity obtaining unit 413 that obtains reception field intensity in wireless communication, or the bit error rate obtaining unit 415 that obtains a wireless communication bit error rate in wireless communication may be applied, instead. Furthermore, the external environment frequency/channel obtaining unit 412 that obtains frequencies and channels in an external environment may be applied. Since it is obvious that the signal transmission time becomes long when the distance to a wireless base station is large, the effect of noise upon an image may be measured more accurately using information regarding the distance along with the signal transmission time, which is included in the scope of the present invention.

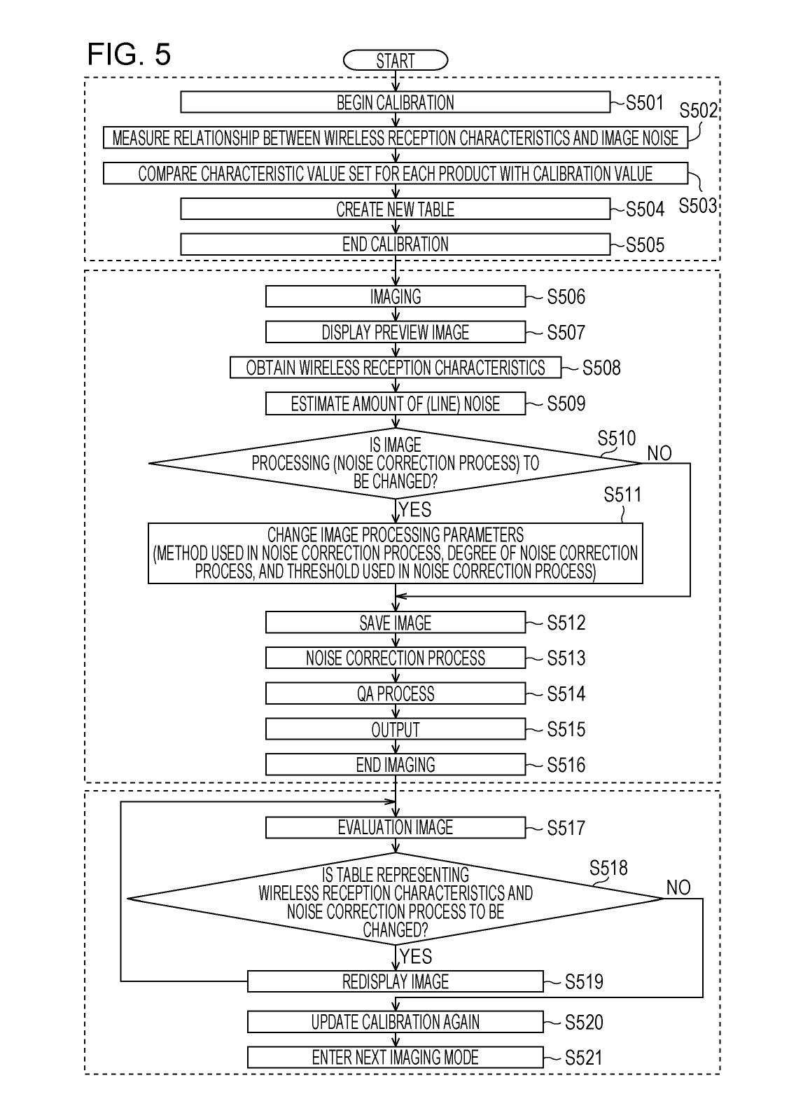

FIG. 5 is a flowchart illustrating an example of a processing procedure of a control method used by an X-ray imaging system (radiation imaging system) 1000 according to the second embodiment of the present invention. In FIG. 5, an example of a processing procedure of a control method used by an X-ray imaging apparatus 1200 is mainly illustrated.

First, in step S501, the calibration unit 420 of the X-ray imaging apparatus 1200 or the like begins to perform calibration. Here, the calibration unit 420 prepares for reduction of image noise in accordance with the wireless reception characteristics based on an external environment or the like by obtaining the wireless reception characteristics and the amount of noise in an image in advance. In order not to allow the external environment to become different between the calibration and the actual imaging, the calibration can be performed immediately before the actual imaging using the same arrangement and the like.

Next, in step S502, the calibration wireless reception characteristics obtaining unit 410 and the calibration image noise measuring unit 430 of the X-ray imaging apparatus 1200 or the like measure the wireless reception characteristics and the image noise, respectively. Unlike in the case of the number of X-rays, it is difficult to use different conditions in the case of the wireless reception characteristics, but when a plurality of conditions are available, the wireless reception characteristics and the image noise are measured under the plurality of conditions. Here, the image noise to be measured includes, for example, the amount of (line) noise, the type of noise, the location of noise, and the frequency of noise.

Next, in step S503, the calibration unit 420 of the X-ray imaging apparatus 1200 or the like compares the characteristic value set for each product with a calibration value.

Next, in step S504, the calibration unit 420 of the X-ray imaging apparatus 1200 creates a new calibration table relating to the wireless reception characteristics and the image noise on the basis of the results obtained in steps S502 and S503 and the like. If the wireless reception characteristics during the imaging may be obtained on the basis of the table obtained as a result of the calibration, it becomes possible to create an estimate of the amount of noise to be superimposed upon the image.

Next, in step S505, the calibration unit 420 of the X-ray imaging apparatus 1200 or the like ends the calibration. Since the external wireless communication environment or the like might be different between the calibration and the imaging, the calibration can be regularly updated. For example, the calibration (steps S501 to S505) may be repeated at intervals of 10 minutes in order to automatically update the calibration table to a latest one.

Next, in step S506, for example, the X-ray imaging system 1000 captures an X-ray image of the subject P. Processing in steps S506 to S516 is the same as that in steps S201 to S211, respectively, illustrated in FIG. 2, and accordingly description thereof is omitted.

Next, in step S517, the X-ray imaging apparatus 1200 or the like evaluates an X-ray image based on X-ray image data output in step S515. An X-ray image used for diagnosis is normally not evaluated. Therefore, it is possible that the X-ray image is not evaluated in order not to change the workflow. If the X-ray image is evaluated, in general, a radiologic technologist or a doctor visually evaluates the X-ray image displayed on the image display unit 1305 or the like. Needless to say, the evaluation includes quantitative evaluation of the X-ray image. The radiological technologist or the doctor examines the displayed X-ray image to obtain the amount of noise that can appear in wireless communication and determine whether or not to reduce the noise through image processing. Because a quantitative value is necessary in order to reduce the noise through the image processing, the X-ray image is evaluated, for example, on a scale of one to five, and information as to whether or not to reduce the noise through the image processing is obtained. Here, for example, “five” is a highest grade and “one” is a lowest grade in the five-grade evaluation.

Next, in step S518, the X-ray imaging apparatus 1200 (the calibration unit 420 or the like) determines whether or not to change a table representing the wireless reception characteristics and the noise correction process on the basis of a result of the evaluation of the image obtained in step S517. For example, if the result of the evaluation of the image obtained in step S517 is smaller than or equal to a certain value (for example, three), the noise is to be reduced through the image processing, and accordingly the table is changed so that the image processing is performed.

If it is determined in step S518 that the table representing the wireless reception characteristics and the noise correction process is to be changed, the procedure proceeds to step S519.

In step S519, the calibration unit 420 of the X-ray imaging apparatus 1200 or the like changes the table representing the wireless reception characteristics and the noise correction process. Thereafter, the X-ray imaging apparatus 1200 performs the noise correction process based on the changed table using the changing unit 1212 and a process for redisplaying an X-ray image based on X-ray image data subjected to the noise correction process.

Attention is required if it is diagnosed that the degree of the noise correction process needs to be decreased. This is because it is generally impossible to tell how much noise is superimposed upon an original image just by seeing an image from which the noise has been reduced, and therefore the image needs to be redisplayed and a loop process needs to be performed several times after the degree of the noise correction process is decreased.

When the X-ray image has been redisplayed in step S519, the procedure returns to step S517, and the X-ray image is evaluated. Thus, by repeating the processing in steps S517 to S519, an appropriate X-ray image according to the needs of each medical institution may be obtained.

If it is determined in step S518 that the table representing the wireless reception characteristics and the noise correction process is not to be changed, the procedure proceeds to step S520.

In step S520, the calibration unit 420 (for example, the calibration result updating unit 426) of the X-ray imaging apparatus 1200 updates calibration information again on the basis of the current table representing the wireless reception characteristics and the noise correction process.

Thereafter, in step S521, the X-ray imaging apparatus 1200 or the like enters a next imaging mode.

The processing procedure of the control method used by the X-ray imaging system 1000 illustrated in FIG. 5 thus ends.

Although an example in which the operation is performed using one calibration image and one X-ray image has been described with reference to the flowchart, a plurality of calibration images and a plurality of X-ray images may be used, instead. For example, obtaining a plurality of images and using a result of calibration obtained by averaging the plurality of images is obviously included in the scope of the present invention. In addition, in this embodiment, differences in the wireless reception characteristics and the amount of noise in an image are calibrated during the calibration. However, the embodiment of the present invention is not limited to performing the calibration first. For example, processing in steps S517 to S519 in which the amount of noise is obtained using a previously captured image and the method (correction method) used in the noise correction process, the degree of the noise correction process, and the like are changed on the basis of the amount of noise is obviously included in the scope of the present invention.

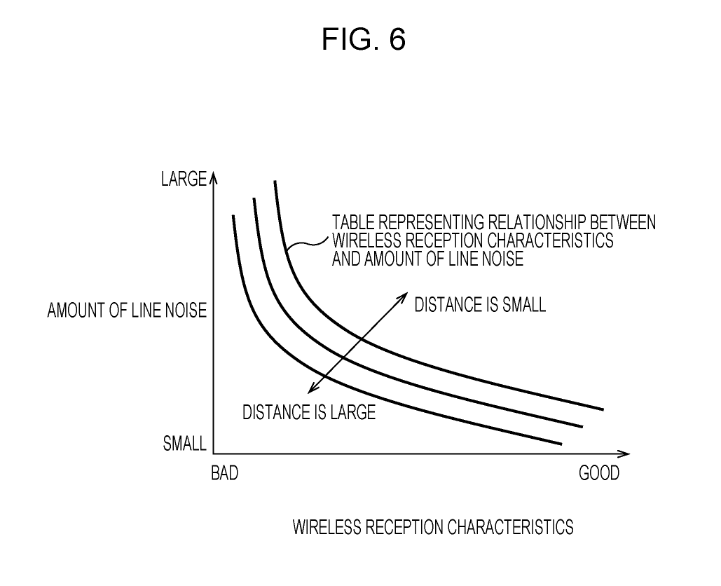

FIG. 6 is a diagram illustrating the calibration according to the second embodiment of the present invention.

As illustrated in FIG. 6, the better the wireless reception characteristics, the smaller (weaker) the amount of line noise, and the worse the wireless reception characteristics, the larger (stronger) the amount of line noise. This relationship varies depending on an installation environment of the X-ray imaging apparatus 1200 and an external wireless state. For example, when a distance between a wireless antenna and a reception unit is large, the amount of line noise is small even if the wireless reception characteristics are weak (bad). This is because of the distance between the wireless communication units (wireless communication apparatuses). Thus, even if the wireless reception characteristics are bad, the amount of line noise to be reduced may be small depending on a result of the calibration.

In the example illustrated in FIG. 6, the calibration table is changed in accordance with the distance between the wireless communication units (wireless communication apparatuses). In addition, for example, the battery management information can also be used for the calibration obviously.

With respect to FIGS. 7A to 7D, in this embodiment, the relationship between the wireless reception characteristics and the amount of line noise may be obtained in advance on the basis of the relationship between the wireless reception characteristics and the amount of line noise during the calibration. Therefore, by changing the degree of the noise correction process, that is, for example, by changing the threshold, a subject image in which the subject P is easy to recognize may be obtained while suppressing a possibility that the subject image is overcorrected.

### Third Embodiment

Next, a third embodiment of the present invention will be described.

The third embodiment is characterized in that wireless reception characteristics when a dark current correction image is obtained are also obtained and used for estimating the amount of noise in an image.

In the X-ray imaging apparatus 1200 including the X-ray sensor 1201 that uses conversion elements (photoelectric conversion elements), at least three images (include correction images) are required in order to generate one image. These correction images are obtained at different timings, and therefore only one set of wireless reception characteristics at the time of X-ray radiation might not be enough for one image. In this embodiment, noise generated when external wireless information constantly changes is reduced.

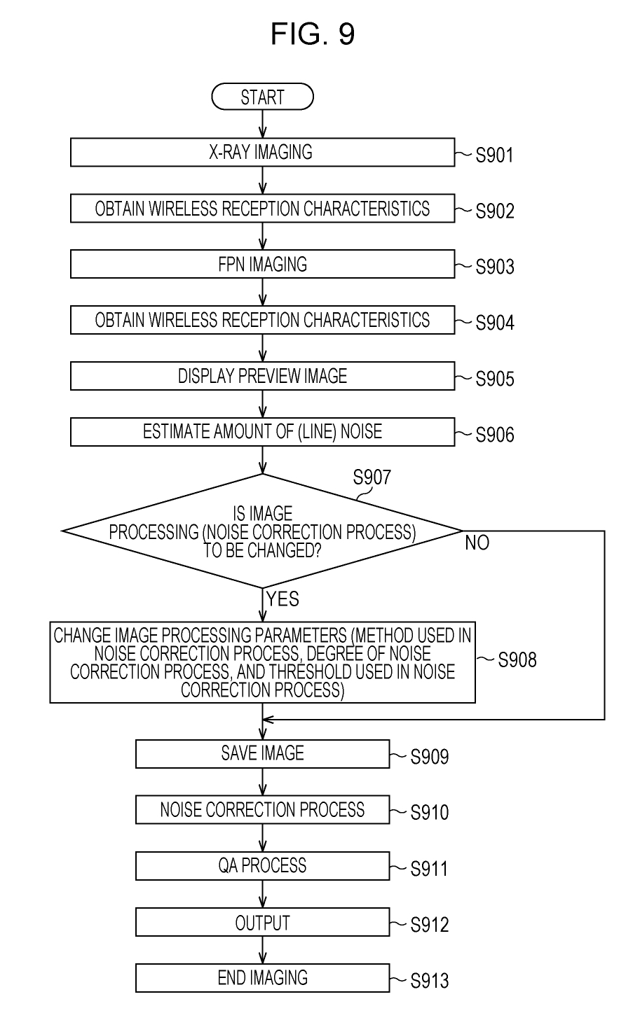

FIG. 9 is a flowchart illustrating an example of a processing procedure of a control method used by an X-ray imaging system (radiation imaging system) 1000 according to a third embodiment of the present invention. In FIG. 9, an example of a processing procedure of a control method used by an X-ray imaging apparatus 1200 according to this embodiment is mainly illustrated.

After obtaining an X-ray image (hereinafter referred to as a “bright image”) through X-ray imaging, the X-ray imaging apparatus 1200 including conversion elements (photoelectric conversion elements) obtains a dark image by performing driving without radiating X-rays in order to correct variations in dark current and a residual image for each pixel. If the wireless reception characteristics are different between the bright image and the dark image, it is difficult to identify the cause after the correction. Therefore, the cause needs to be identified using noise in the image before the correction.

Therefore, first, in step S901, the X-ray imaging system 1000 performs X-ray imaging of the subject P. In the flowchart of FIG. 2 according to the first embodiment and the flowchart of FIG. 5 according to the second embodiment, a case in which a bright image and a dark image are simultaneously obtained (S201 and S506) is assumed. In the third embodiment, however, a bright image, which is an image obtained by capturing a frame image while X-rays are being radiated, and a dark image, which is a frame image for correcting dark current obtained while X-rays are not being radiated, are obtained at different timings. In this case, an image after the correction of the dark current may be obtained by subtracting the dark image from the bright image, that is, X−F (bright image−dark image).

In the X-ray imaging, pixels of an X-ray sensor 1201 accumulate electrical signals (image signals) based on the X-rays that have passed through the subject P, and the electrical signals are sequentially read by switching elements including TFTs. Thereafter, pixel signals subjected to A/D conversion and amplification performed by an amplifier are sequentially arranged, thereby generating a bright image, which is an X-ray frame image.

Since a period of time immediately before the pixel signals are amplified by the amplifier is a period of time in which noise is most likely to be superimposed, a characteristics obtaining unit 1213 of the X-ray imaging apparatus 1200 obtains the wireless reception characteristics of a wireless communication unit 1217 in this period of time in step S902. Although the wireless reception characteristics are obtained in a period of time before the amplification performed by the amplifier but after the X-ray imaging in this embodiment, the scope of the present invention is not limited to this. That is, the processing in step S902 may be performed immediately after or at the same time as the reading of the image during the X-ray imaging in step S901.

Next, in step S903, the X-ray imaging system 1000 captures a dark image for correcting dark current while X-rays are not being radiated.

In general, in the X-ray imaging apparatus 1200 including the conversion elements (photoelectric conversion elements), variations in the dark current and offsets caused by the dark current are inevitably generated in the pixels. Therefore, correction of dark current is often performed using a dark image obtained in advance or a dark image captured after a bright image is obtained. If the phase and the amount of noise are the same between the dark image obtained in step S903 and the bright image obtained in step S901, only the correction of dark current may be performed, but in an external environment of wireless communication or the like applied in the present invention, it is highly unlikely that the phase and the amount of phase are the same between the dark image and the bright image. In particular, when a dark image obtained in advance is used, there might be significant differences in time and space, and accordingly the wireless reception characteristic at the time of the correction can be obtained.

Therefore, in step S904, the characteristics obtaining unit 1213 of the X-ray imaging apparatus 1200 obtains the wireless reception characteristics of the wireless communication unit 1217 at a time when the dark current is obtained in step S903.

Steps S905 to S913 are the same as step S202 and steps S204 to S211, respectively, illustrated in FIG. 2, and accordingly description thereof is omitted.

In this embodiment, the wireless reception characteristics are obtained not only when the bright image is obtained but also when the dark image is obtained. Although the dark image is obtained after the bright image is obtained in the example illustrated in the flowchart of FIG. 9, this embodiment is not limited to this. For example, an X-ray imaging apparatus 1200 that obtains a dark image in advance and that, when a bright image is obtained, performs dark current correction using the dark image obtained in advance is obviously included in the scope of the present invention.

In another embodiment, the preprocessing unit 1205, the image processing unit 1206, the determination unit 1211, and the changing unit 1212 may be provided for an apparatus different from an apparatus including the X-ray sensor 1201. For example, the X-ray imaging apparatus 1200 may be divided into an imaging apparatus including the X-ray sensor 1201, the A/D conversion unit 1202, the amplification unit 1203, and the data collection unit 1204 and an image processing apparatus including the other components, and a wireless communication unit may be provided for the imaging apparatus. In this case, the wireless communication unit in the imaging apparatus wirelessly communicates with the wireless communication control unit 1214 and the wireless communication unit 1217 in the image processing apparatus. By using such a configuration, the size of the imaging apparatus, which needs to be provided close to a subject, may be reduced, and image processing and display may be performed by the image processing apparatus. Such a system configuration is effective when, for example, the imaging apparatus is portable.

### Other Embodiments

The present invention may also be realized by executing the following process. That is, the present invention may also be realized by executing a process for supplying software (program) that realizes the functions according to one of the above-described embodiments to a system or an apparatus through a network or one of various types of storage media and causing a computer (or a CPU, a microprocessor unit (MPU), or the like) to read and execute the program.

The program and a computer-readable recording medium storing the program are included in the scope of the present invention.

The above-described embodiments of the present invention are merely specific examples for implementing the present invention, and shall not be interpreted as restricting the technical scope of the present invention. That is, the present invention may be implemented in various ways without deviating from the technical spirit and the principal characteristics thereof.

According to the embodiments of the present invention described above as examples, it is possible to obtain a radiation image in which noise is suppressed even while a wireless communication unit is performing wireless communication.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-255426 filed Nov. 21, 2012, which is hereby incorporated by reference herein in its entirety.

What is claimed is:

1. A radiation imaging apparatus comprising:
a radiation imaging unit configured to capture a radiation image of a subject;
a wireless communication unit configured to perform wireless communication with an external apparatus;
a wireless reception characteristics obtaining unit configured to obtain a wireless reception characteristic of the wireless communication unit;
an estimation unit configured to estimate an amount of noise included in the radiation image on the basis of a characteristic value of the wireless reception characteristic; and
a correction unit configured to correct the noise included in the radiation image on the basis of the amount of noise estimated by the estimation unit.

2. The radiation imaging apparatus according to claim 1,
wherein the estimation unit estimates the amount of noise included in the radiation image if the characteristic value of the wireless reception characteristic is smaller than a certain value.

3. The radiation imaging apparatus according to claim 1,
wherein the correction unit performs a first noise correction process if the characteristic value of the wireless reception characteristic is larger than a certain value and performs a second noise correction process based on the estimated amount of noise if the characteristic value of the wireless reception characteristic is smaller than the certain value.

4. The radiation imaging apparatus according to claim 1,
wherein the estimation unit does not perform a process for estimating the amount of noise if the characteristic value of the wireless reception characteristic is larger than a certain value.

5. The radiation imaging apparatus according to claim 1, further comprising:
a changing unit configured to increase a degree of the noise correction process as the amount of noise increases.

6. The radiation imaging apparatus according to claim 1, further comprising:
a calibration unit configured to calibrate a relationship between the wireless reception characteristic and the amount of noise.

7. The radiation imaging apparatus according to claim 6, further comprising:
a distance obtaining unit configured to obtain a distance between the wireless communication unit and a wireless communication unit included in the external apparatus,
wherein the calibration unit performs the calibration using distance information regarding the distance obtained by the distance obtaining unit.

8. The radiation imaging apparatus according to claim 6, further comprising:
a battery management information obtaining unit configured to obtain battery management information regarding the radiation imaging apparatus,
wherein the calibration unit performs the calibration using the battery management information.

9. The radiation imaging apparatus according to claim 6, further comprising:
at least one of the following obtaining units, that is, a frequency/channel obtaining unit configured to obtain a frequency and a channel in an external environment, a reception field intensity obtaining unit configured to obtain reception field intensity in the wireless communication, a signal transmission time obtaining unit configured to obtain signal transmission time in the wireless communication, and a bit error rate obtaining unit configured to obtain a wireless communication bit error rate relating to the wireless communication,
wherein the calibration unit performs the calibration using information obtained by the at least one obtaining unit.

10. The radiation imaging apparatus according to claim 5:
wherein the estimation unit estimates the amount of noise in each region of the radiation image, and
wherein the changing unit changes the degree of the noise correction process for each region of the radiation image.

11. The radiation imaging apparatus according to claim 5,
wherein the changing unit changes at least a correction method used in the noise correction process, the degree of the noise correction process, or a threshold used in the noise correction process.

12. An image processing apparatus that processes a radiation image obtained by a radiation imaging apparatus which wirelessly communicates with an external apparatus, the image processing apparatus comprising:
an obtaining unit configured to obtain a wireless reception characteristic of the radiation imaging apparatus;
an estimation unit configured to estimate an amount of noise included in the radiation image on the basis of a characteristic value of the wireless reception characteristic; and
a correction unit configured to correct the noise included in the radiation image on the basis of the amount of noise estimated by the estimation unit.

13. A method for processing a radiation image obtained by a radiation imaging apparatus that wirelessly communicates with an external apparatus, the method comprising the steps of:
obtaining a wireless reception characteristic of the radiation imaging apparatus;
estimating an amount of noise included in the radiation image on the basis of a characteristic value of the wireless reception characteristic; and
correcting the noise included in the radiation image on the basis of the amount of noise estimated by the estimation unit.

14. A nonvolatile recording medium storing a program for causing a computer to execute the method according to claim 13.
